(19) United States
(12) Patent Application Publication
(10) Pub. No.: US 2012/0296424 A1
(43) Pub. Date: Nov. 22, 2012

(54) ACCOMODATING INTRAOCULAR LENS

(76) Inventor: Nir Betser, Yehud (IL)

(21) Appl. No.: 13/522,981

(22) PCT Filed: Jan. 24, 2011
(86) PCT No.: PCT/US2011/022188

§ 371 Date: Jul. 19, 2012

Related U.S. Application Data
(60) Provisional application No. 61/298,219, filed on Jan. 26, 2010.

(51) Int. Cl. A61F 2/16

(52) U.S. Cl. 623/6.13

(57) ABSTRACT

An accommodating intraocular lens (AIOL) (10) characterised by a first member (1) and a second member (3) arranged to correspond to anterior and posterior portions of a capsular bag, having an anterior-posterior axis passing centrally through the first and second members (1, 3), the first and second members (1, 3) being connected by one or more link members (2), a lens structure (4) including an inflatable lens (21), and a reservoir (23) of filling fluid (12) in fluid communication with the inflatable lens (21) through one or more channels (22), wherein axial movement of one of the first and second members (1, 3) along the anterior-posterior axis applies a pumping force to cause the filling fluid (12) to flow between the reservoir (23) and the inflatable lens (21).

### FIELD OF THE INVENTION

The present invention generally relates to accommodating intraocular lenses.

### BACKGROUND OF THE INVENTION

Intraocular lenses (IOLs) have been in use for more than 60 years as an implanted replacement for the natural lens in the human eye after cataract surgery. Until about the age of 40, the natural lens can change its curvature shape, and as a result its optical power, for sharp vision of far and near objects in a process called accommodation.

Many ideas for accommodating intraocular lenses (AIOLs) have been proposed in recent years but none of them attain the required level of accommodation to enable vision without glasses after cataract surgery.

### SUMMARY OF THE INVENTION

The present invention seeks to provide an improved accommodating intraocular lens, as is described more in detail hereinbelow.

There is provided in accordance with an embodiment of the present invention an accommodating intraocular lens (AIOL) including an anterior haptic member and a posterior haptic member arranged to correspond to anterior and posterior portions of a capsular bag, having an anterior-posterior axis passing centrally through the anterior and posterior haptic members, an optic including an inflatable member, and a reservoir of fluid in fluid communication with at least one of the anterior and posterior haptic members and with the inflatable member, wherein axial movement of one of the anterior and posterior haptic members along the anterior-posterior axis applies a pumping force to cause fluid to flow between the reservoir and the inflatable member.

### BRIEF DESCRIPTION OF THE DRAWINGS

The present invention will be understood and appreciated more fully from the following detailed description taken in conjunction with the drawings in which:

FIGS. 1A and 1B are simplified perspective illustrations of an accommodating intraocular lens (AIOL), constructed and operative in accordance with an embodiment of the present invention, respectively in an accommodated state (near vision) with a membrane inflated, and an un-accommodated state (far vision) with the membrane deflated;

FIGS. 2A and 2B are simplified side illustrations of the AIOL, in respective accommodated and un-accommodated states in the capsular bag;

FIGS. 3A-3G are simplified perspective, top, perspective, side, side sectional, side and top illustrations, respectively, of the lens structure of the AIOL, in accordance with an embodiment of the present invention;

FIG. 4 is a simplified sectional illustration of the frame and lens structure of the AIOL, in accordance with an embodiment of the present invention;

FIGS. 5A-5D are simplified front, side sectional, top and perspective illustrations, respectively, of the AIOL, in the accommodated state;

FIGS. 5E-5H are simplified front, side, top and perspective illustrations, respectively, of the AIOL, in the un-accommodated state;

FIGS. 6A-6D are simplified front, side sectional, top and perspective illustrations, respectively, of the AIOL, in the accommodated state, in accordance with another embodiment of the present invention;

FIGS. 6E-6H are simplified front, side sectional, top and perspective illustrations, respectively, of the AIOL, in the un-accommodated state, in accordance with another embodiment of the present invention;

FIGS. 7A-7D and 8A-8D are simplified front, side sectional, top and perspective illustrations, respectively, of other accommodating intraocular lenses, constructed and operative in accordance with other embodiments of the present invention;

FIGS. 9A-9B are simplified top illustrations of an accommodating intraocular lens, constructed and operative in accordance with an embodiment of the present invention, with two different alternative ring (haptic) structures;

FIGS. 10A-10F are simplified front, side sectional, top, perspective, front and side sectional illustrations, respectively, of accommodating intraocular lenses, constructed and operative in accordance with other embodiments of the present invention, wherein the AIOL is placeable on top of another non-accommodating IOL;

FIGS. 11A-11D are simplified front, side, top and perspective illustrations, respectively, of an AIOL, in the accommodated state, in accordance with another embodiment of the present invention;

FIGS. 12A-12D are simplified front, side, top and perspective illustrations, respectively, of the AIOL of FIGS. 11A-11D, in the un-accommodated state;

FIGS. 13A-13D are simplified top, perspective, front and side illustrations, respectively, of the AIOL of FIGS. 11A-11D, installed in an eye in the accommodated state;

FIGS. 14A-14D are simplified top, perspective, front and side illustrations, respectively, of the AIOL of FIGS. 11A-11D, installed in an eye in the un-accommodated state;

FIGS. 15A-15B are simplified side and sectional illustrations, respectively, of the AIOL of FIGS. 11A-11D, in the un-accommodated state;

FIGS. 16A-16D are simplified front, side, top and perspective illustrations, respectively, of the lens structure, includes a solid lens and an inflatable lens (membrane), of the AIOL of FIGS. 11A-11D, in the un-accommodated state;

FIGS. 17A-17D are simplified front, side, top and perspective illustrations, respectively, of an AIOL, in the accommodated state, in accordance with yet another embodiment of the present invention;

FIGS. 18A-18D are simplified front, side, top and perspective illustrations, respectively, of the AIOL of FIGS. 17A-17D, in the un-accommodated state;

FIGS. 19A-19D are simplified front, side, top and perspective illustrations, respectively, of an AIOL, in the accommodated state, in accordance with still another embodiment of the present invention; and

FIGS. 20A-20D are simplified front, side, top and perspective illustrations, respectively, of the AIOL of FIGS. 19A-19D, in the un-accommodated state.

### DETAILED DESCRIPTION OF EMBODIMENTS

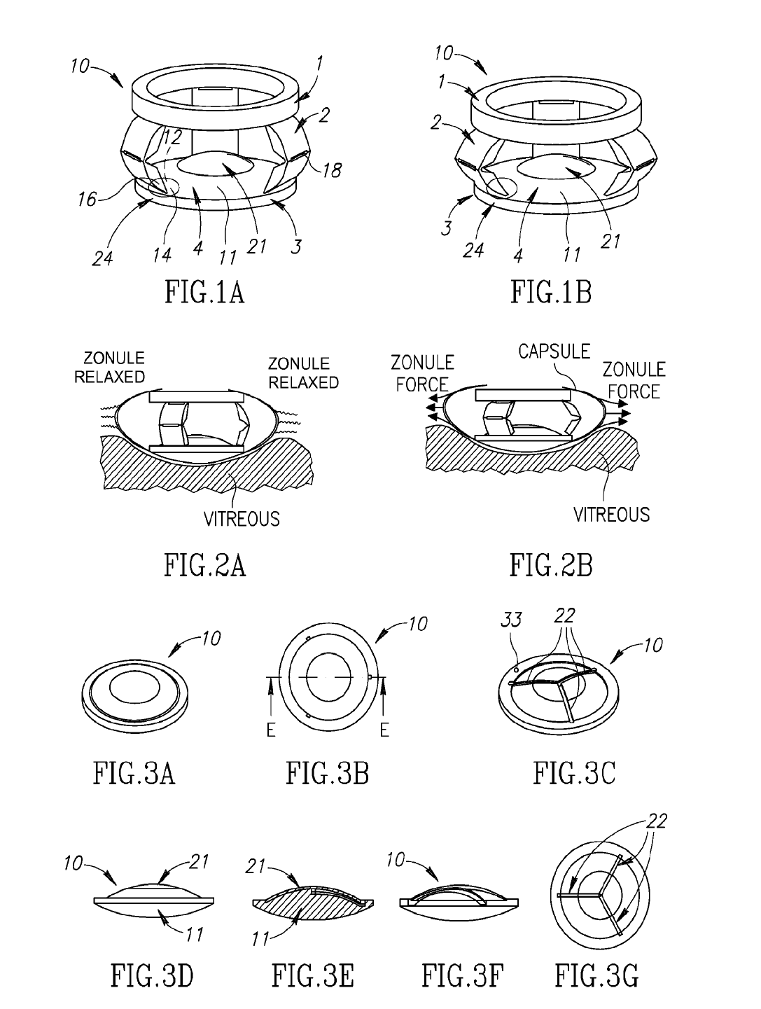

Reference is now made to FIGS. 1A and 1B, which illustrate an accommodating intraocular lens (AIOL) 10, constructed and operative in accordance with an embodiment of the present invention.

The AIOL 10 includes a frame structure constructed of a first portion 1 and a second portion 3 connected to one another by one or more link members 2. First and second portions 1 and 3 are adapted to sit in the anterior and posterior portions, respectively, of the capsular bag (not shown) after removal of the natural lens. (Alternatively the first portion can be the posterior portion and the second portion can be the anterior portion of the AIOL as installed in the eye.) First and second portions 1 and 3 serve as the haptics the hold the AIOL 10 in the bag; alternatively other haptic structures, such as curved wires or plate haptics, for example, may be added to protrude from first and second portions 1 and 3.

The second portion 3 includes a lens structure 4 that includes a solid lens 11 with the required optical power to reach clear vision and an inflatable lens (membrane) 21 (e.g., about 2-3 mm in diameter), preferably, but not necessarily, at the central part of the lens 11. Alternatively, lens structure 4 can include just the inflatable lens 21.

Inflatable lens 21 is constructed of a material with sufficient resilience that enables it to expand and increase its convexity upon filling with a filling fluid and contract and decrease its convexity upon evacuation therefrom of the filling fluid. Solid lens 11 is preferably stiffer than inflatable lens 21, but alternatively, can be of the same stiffness as inflatable lens 21. The term “stiffness” refers to the amount of elastic deformation a material undergoes when subjected to a given amount of force: the less elastic deformation the material undergoes due to a given force, the stiffer the material. Lens 21 may be spheric, aspheric, toric or other types of optics.

In one embodiment of the invention, solid lens 11 and inflatable lens 21 are both made of materials that belong to the same class of polymeric materials and are derived from monomers which are mutually compatible, allowing the materials to be co-cured and/or bonded, for example chemically bonded or otherwise joined, to one another. For example, these materials include, without limitation, acrylic polymeric materials, cross-linked acrylic materials, copolymers of methacrylate and acrylate esters cross-linked with one or more functional acrylate/methacrylate cross-linking components, hydrogels, (e.g., hydroxyethyl methacrylate (HEMA) polymer or methyl methacrylate/N-vinyl pyrrolidone (MMA/NVP) copolymer or the like), silicon-containing polymeric materials, such as hydrophobic and hydrophilic silicone, and others.

In another embodiment, solid lens 11 may be constructed of a different material than inflatable lens 21, such as but not limited to, polymethylmethacrylate (PMMA), collagen, hydrogel, hyaluronic acid, polysulfones, thermolabile materials and other relatively hard or relatively soft and flexible biologically inert optical materials.

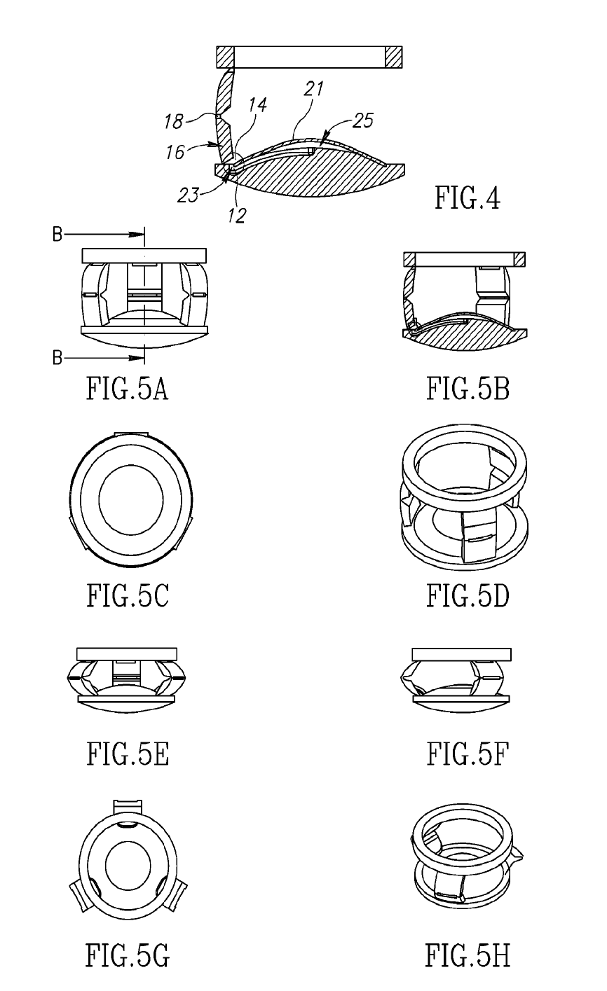

A reservoir 23 containing filling fluid 12 (such as but not limited to, water, saline solution, oil, silicone oil and other medically approved liquids, air or other gas, gel or others) is located at the periphery of second portion 3. A chamber 25 (FIG. 4) is located between solid lens 11 and inflatable lens 21. One or more channels 22 fluidly connect reservoir 23 to chamber 25. A resilient pedal 14 overlies an anterior portion of reservoir 23. Pedal 14 can be a pad located on top of reservoir 23 or a membrane covering reservoir 23, for example, made of a resilient material or relatively stiffer material or combination thereof. A leg 16 at an end of link member 2 near the second portion 3 is positioned over pedal 14. Leg 16 of link member 2 and pedal 14 together form a pumping device 24 to pump filling fluid 12 to inflate inflatable lens 21, as is now described. Reservoir 23 is also considered part of the pumping device 24. Other pumping devices may also be used, examples of which are described further below. Lens 21 may be sufficiently stiff to apply a force on the filling fluid 12 to cause the filling fluid 12 to flow back to reservoir 23.

AIOL 10 fills the capsular bag and restores it or nearly restores it, to its volumetric state before removal of the natural lens. Without wishing to be limited to any particular theory of operation, it is believed that accommodative forces are exerted on AIOL 10 by the zonules, ciliary muscles, and capsular bag. These accommodative forces cause axial translation (i.e., along the anterior-posterior directions) of the first and second portions 1 and 3 (that is, first portion 1 moves towards or away from second portion 3, or second portion 3 moves towards or away from first portion 1, or a combination of movement towards or away from each other).

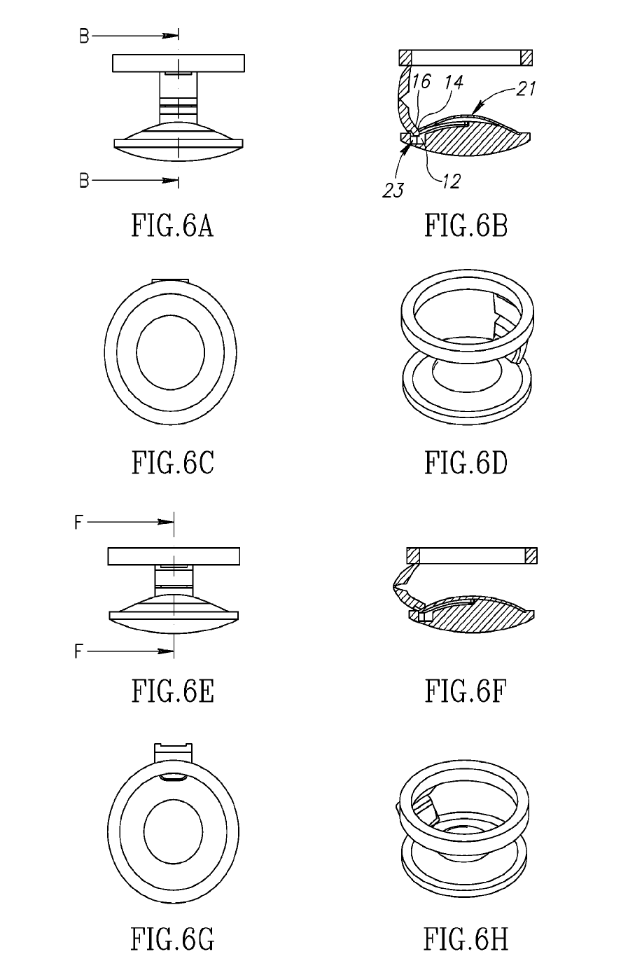

In the position shown in FIGS. 1A, 2A, 4, 5A-5D and 6A, the first and second portions 1 and 3 are axially further from one another. The relative axial translation of the first and second portions 1 and 3 away from each other causes link members 2 to become straightened or at least less bent. As link member 2 straightens (becomes less bent), the leg 16 of link member 2 pushes on pedal 14 and pumps the filling fluid 12 out of reservoir 23 through channels 22 to chamber 25 to inflate (expand) inflatable lens 21. This is the position for focusing on near objects (near vision).

Conversely (as seen in FIGS. 1B, 2B, 5E-5H and 6B), relative axial translation of the first and second portions 1 and 3 towards each other causes flexure (bending) of link members 2, such as at hinges 18 formed in link members 2. (Without being bound by any theory, this may occur upon contraction of the capsular bag due to ciliary muscle relaxation, which stretches the capsular bag towards the capsule equator and decrease the distance between the poles.) As link member 2 flexes (bends), the leg 16 of link member 2 comes off pedal 14 Filling fluid 12 flows out of chamber 25 through channels 22 to reservoir 23, thereby deflating inflatable lens 21. This is the position for focusing on far objects (far vision).

Thus, the optical power of the center part of the combined structure is altered and increased by extra diopters. These extra diopters add to the lens power and enable the patient to have sharp near vision. The lens structure of the invention can have the required optical power for sharp distance vision for a patient undergoing refracting lens exchange (RLE) usually as part of cataract surgery. Lens power may be, without limitation, around +20 diopters.

It is noted that since during the accommodation process the pupil diameter decreases, it may be sufficient to limit the curvature change of lens 21 over a sub-portion of the lens surface with a diameter of about 2-3 mm and no need to make the curvature change over the entire lens surface of lens 21, which may typically be about 4-6 mm This is a huge advantage that simplifies the design, however, the invention is not limited to this sub-portion of the lens surface.

In the non-accommodating state (far vision) the membrane of inflatable lens 21 may have almost the same curvature as the anterior surface of the solid lens 11.

A port 33 (shown optionally in FIG. 3C, but applicable for all embodiments) may be provided for filling the reservoir 23 with the filling fluid 12 during production and/or in another procedure, also allowing power adjustment and/or refilling. AIOL 10 can have different devices and/or structures for altering power of both lenses prior, during and after implantation.

Two or more lens can be provided in the structure for obtaining different optical effects. Any number of channels and link members may be used, with different shapes and positions. The solid lens 11 and inflatable lens 21 may have the same index of refraction, or alternatively, different indices of refraction.

The invention eliminates the risk of having liquid in a chamber in the eye since a very small volume of filling fluid is needed (e.g., about 0.5 mm³) for creating the accommodation.

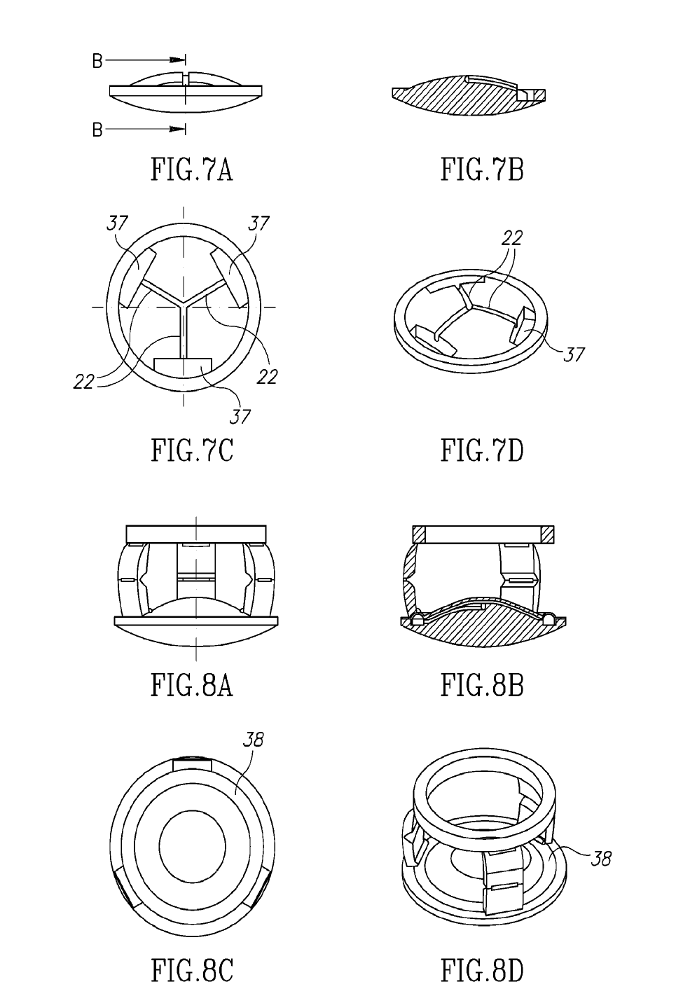

Reference is now made to FIGS. 7A-7D and 8A-8D, which illustrate other accommodating intraocular lenses, constructed and operative in accordance with other embodiments of the present invention. The operating principles are the same as described above. In the embodiment of FIGS. 7A-7D, there are three separate reservoirs 37 spaced equally apart. In the embodiment of FIGS. 8A-8D, the reservoir is a single annular reservoir 38.

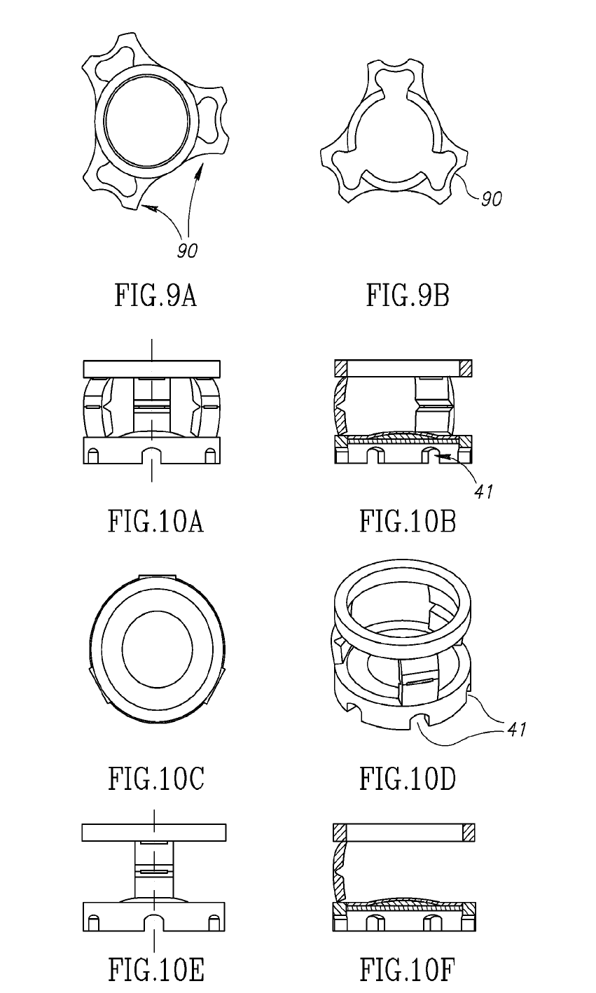

Reference is now made to FIGS. 9A-9B, which illustrate an accommodating intraocular lens, constructed and operative in accordance with an embodiment of the present invention. This embodiment has three axi-symmetrical winged haptics 90 extending from either or both of first and second portions of the AIOL. These haptics may help support the anterior and posterior parts of the capsular bag to retain the desired shape of the bag. This may help exploit the forces and movements of the eye structure during the accommodation process. Of course, the invention may include any number of haptics, winged or not.

Reference is now made to FIGS. 10A-10F, which illustrate accommodating intraocular lenses, constructed and operative in accordance with other embodiments of the present invention. In this embodiment, grooves (openings) 41 are formed on a surface of one or both of the first and second portions of the AIOL. This enables placing the AIOL on top of another non-accommodating IOL.

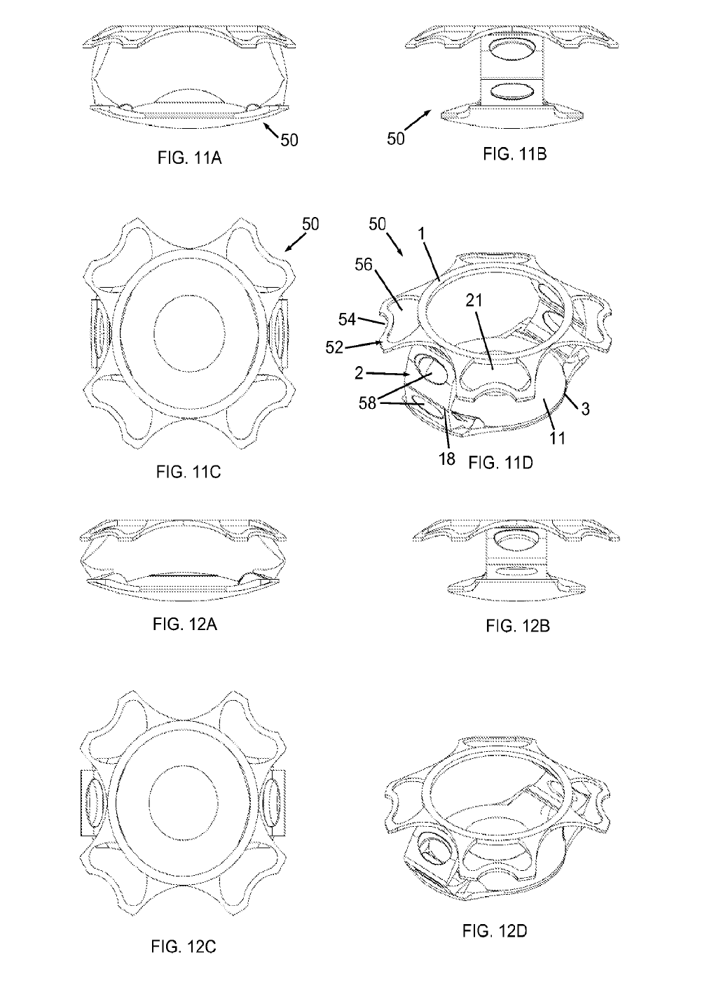

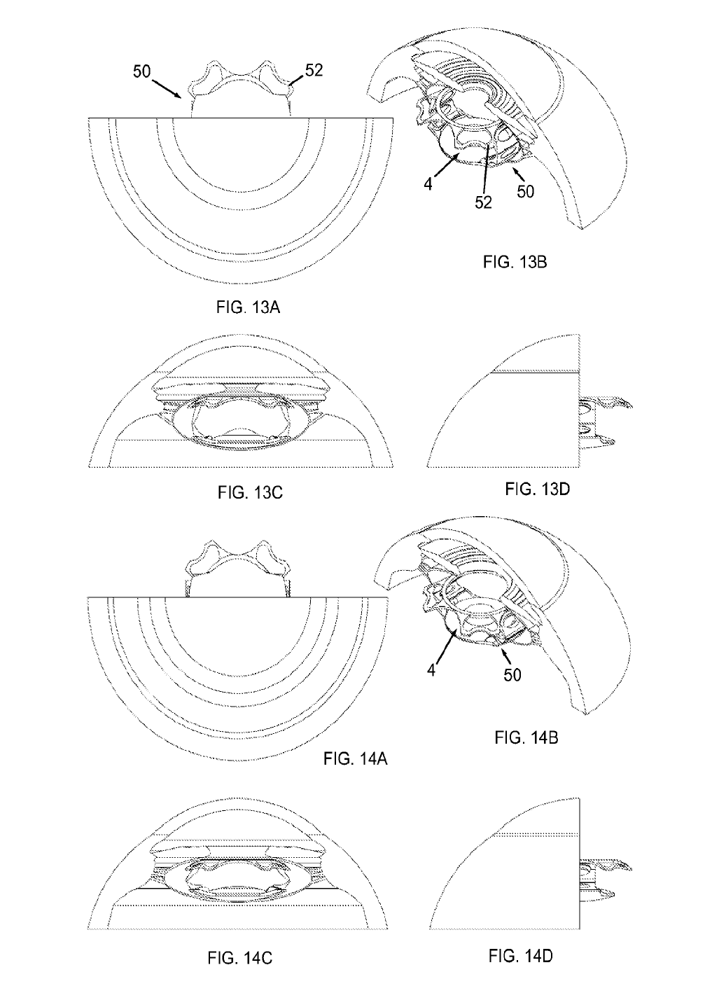

Reference is now made to FIGS. 11A-15B, which illustrate an AIOL 50, constructed and operative in accordance with another embodiment of the present invention. Like elements are labeled with like numerals. This embodiment has four (any other number is also possible) axi-symmetrical winged haptics 52 extending from first portion 1 of the AIOL 50. Additionally or alternatively they could extend from the second portion 3. Haptic 52 has a radially-outward curved contour 54 for better matching the shape of the capsular bag. Haptic 52 has a cutout portion 56 to reduce weight and so as not to block tissue in the eye. Link members 2 also have cutout portions 58 formed therein on either side of hinge 18.

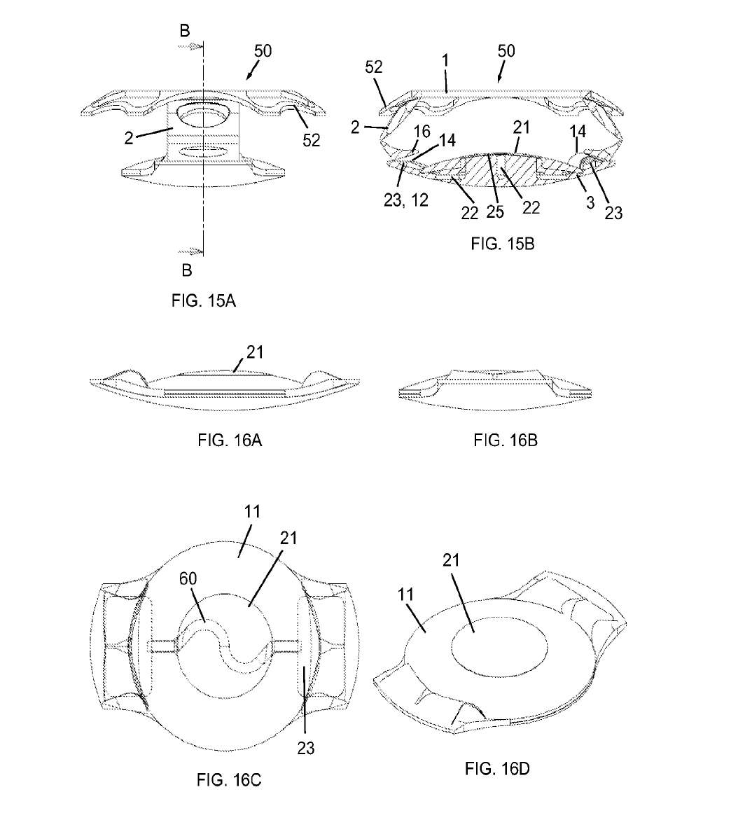

Reference is now made to FIGS. 16A-16D, which illustrate the lens structure of the embodiment of FIGS. 11A-15B. In this embodiment, there are serpentine or S-shaped channels 60. Other shapes may also be used.

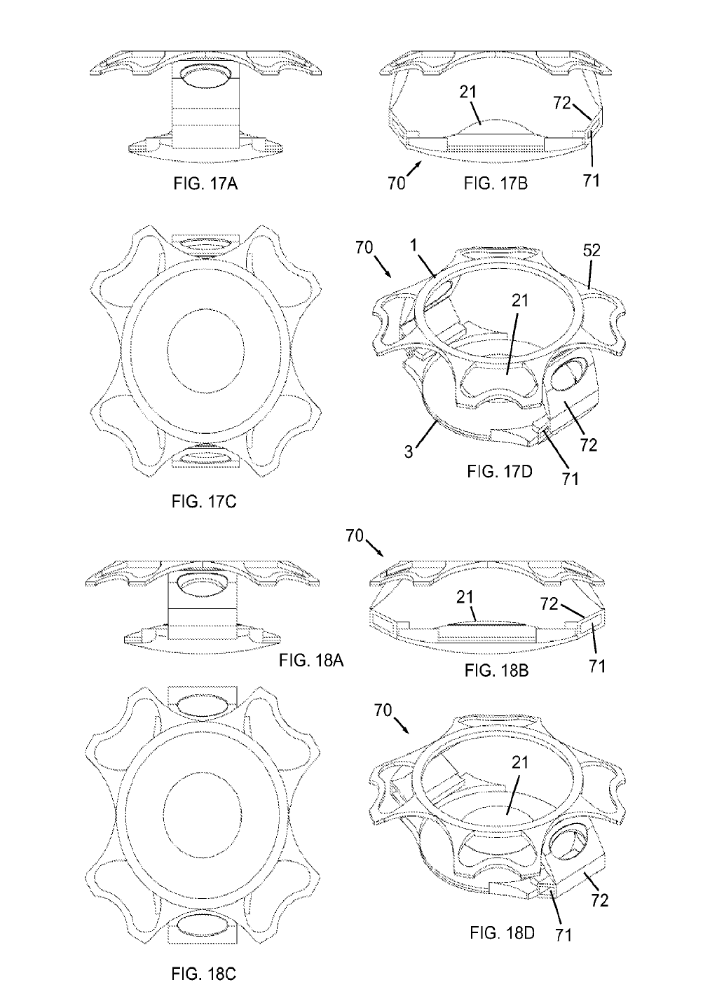

Reference is now made to FIGS. 17A-18D, which illustrate an AIOL 70, constructed and operative in accordance with yet another embodiment of the present invention. In this embodiment, there is a reservoir 71 in the leg (lower leg) of one or more of link members 72, and reservoir 71 contracts and expands with movement of the link members 72, which is the pumping action. When the first and second portions 1 and 3 are moved away from one another for near vision, the filling fluid flows from reservoir 71 to inflatable lens 21; reservoir 71 is squeezed (contracted) by this action, thereby pumping fluid from it to the lens 21. Conversely, when the first and second portions 1 and 3 are moved towards one another for far vision, the filling fluid flows from inflatable lens 21 to reservoir 71; reservoir 71 expands as it is filled with the fluid flowing from lens 21.

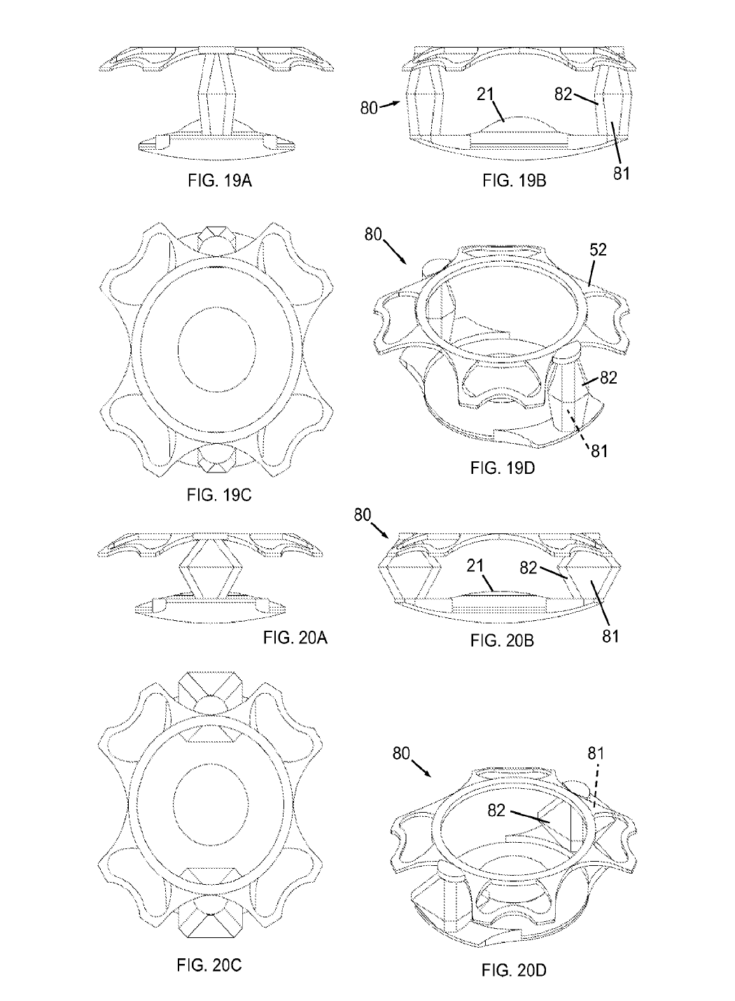

Reference is now made to FIGS. 19A-20D, which illustrate an AIOL 80, constructed and operative in accordance with still another embodiment of the present invention. In this embodiment, there is a reservoir 81 formed in an inner space of a link member 82, and reservoir 81 contracts and expands with movement (flexure) of the link members 82, which is the pumping action. When the first and second portions 1 and 3 are moved away from one another for near vision, the filling fluid flows from reservoir 81 to inflatable lens 21; reservoir 81 is contracted by this action, thereby pumping fluid from it to the lens 21. Conversely, when the first and second portions 1 and 3 are moved towards one another for far vision, the filling fluid flows from inflatable lens 21 to reservoir 81; reservoir 81 expands as it is filled with the fluid flowing from lens 21.

It will be appreciated by persons skilled in the art that the present invention is not limited by what has been particularly shown and described hereinabove. Rather the scope of the present invention includes both combinations and subcombinations of the features described hereinabove as well as modifications and variations thereof which would occur to a person of skill in the art upon reading the foregoing description and which are not in the prior art.

1. An accommodating intraocular lens (AIOL) comprising:
a first member and a second member arranged to correspond to anterior and posterior portions of a capsular bag, having an anterior-posterior axis passing centrally through said first and second members, said first and second members being connected by one or more link members;
a lens structure comprising an inflatable lens; and
a reservoir of filling fluid in fluid communication with said inflatable lens through one or more channels,
wherein a pumping device is operative to employ axial movement of one of said first and second members along the anterior-posterior axis to apply a pumping force to cause said filling fluid to flow between said reservoir and said inflatable lens.

2. The AIOL according to claim 1, wherein said pumping device pumps fluid from said reservoir to said inflatable lens.

3. The AIOL according to claim 1, wherein said link member is bendable about a hinge.

4. The AIOL according to claim 1, wherein said lens structure further comprises a solid lens on which said inflatable lens is located.

5. The AIOL according to claim 4, wherein said inflatable lens is centrally located on said solid lens.

6. The AIOL according to claim 4, wherein a chamber is located between said solid lens and said inflatable lens and said one or more channels, fluidly connect said reservoir to said chamber.

7. The AIOL according to claim 1, wherein said pumping device comprises a resilient pedal that overlies a portion of said reservoir and a leg at an end of said link member near said second portion is positioned over said pedal, wherein axial movement of one of said first and second members along the anterior-posterior axis causes said leg to press against said pedal to pump said filling fluid from said reservoir to said inflatable lens.

8. The AIOL according to claim 1, further comprising a port for filling said reservoir with said filling fluid.

9. The AIOL according to claim 1, wherein said reservoir comprises reservoirs spaced equally apart.

10. The AIOL according to claim 1, wherein said reservoir comprises a single annular reservoir.

11. The AIOL according to claim 1, wherein grooves are formed on a surface of one or both of said first and second portions.

12. The AIOL according to claim 1, comprising axi-symmetrical winged haptics extending from at least one of said first and second portions.

13. The AIOL according to claim 12, wherein said haptics have a radially-outward curved contour and a cutout portion.

14. The AIOL according to claim 1, wherein said pumping device comprises said reservoir being located in a leg of said link member, and said reservoir contracts and expands with movement of said link member.

15. The AIOL according to claim 1, wherein said pumping device comprises said reservoir being located in an inner space of said link member, and said reservoir contracts and expands with movement of said link member.
